(19) United States
(12) Patent Application Publication
(10) Pub. No.: US 2015/0099715 A1
(43) Pub. Date: Apr. 9, 2015

(54) FORMULATIONS OF ACADESINE

(71) Applicant: Merck Sharp & Dohme Corp., Rahway, NJ (US)

(72) Inventors: Valentyn Antochshuk, Summit, NJ (US); Mohammed Shameem, Kenilworth, NJ (US); Amadeep Singh Bhupender BHALLA, Summit, NJ (US)

(73) Assignee: MERCK SHARP & DOHME CORP., Rahway, NJ (US)

(21) Appl. No.: 14/570,385

(22) Filed: Dec. 15, 2014

Related U.S. Application Data
(63) Continuation of application No. 13/515,332, filed on Jun. 12, 2012.
(60) Provisional application No. 61/286,564, filed on Dec. 15, 2009.

(51) Int. Cl. A61K 31/7056; A61K 47/10; A61K 47/02; A61K 47/18

(52) U.S. Cl. CPC A61K 31/7056; A61K 47/18; A61K 47/10; A61K 47/02
USPC 514/43

(57) ABSTRACT

This invention provides a buffered solution comprising acadesine, as well as compositions, formulations and kits containing said solution. The solution, compositions, formulation, and kits can be used to prevent morbidity/mortality in a patient or to treat an ischaemic condition, a condition regulated by adenosine, or a condition associated with reduced blood flow to a tissue in a patient.

### FIELD OF THE INVENTION

The present invention provides formulations of acadesine that are stable and substantially free of discoloration problems.

### BACKGROUND OF THE INVENTION

Ischemia is a low oxygen state typically caused by inadequate blood flow to a tissue resulting in reduced oxygen supply to the tissue, or hypoxia. Several types of ischemia exist including myocardial, mesenteric, and cerebral. It is known that the purine nucleoside, adenosine, can exert a protective effect under ischemic conditions.

Acadesine or 5-aminoimidazole-4-carboxamide (AICA) riboside, a precursor molecule of nucleotide biosynthesis, can enhance the local endogenous levels of extracellular adenosine during periods of ischemia.

PCT Publication WO 2007/044357 relates to formulations comprising acadesine, including buffered solutions of acadesine. However, this document is completely silent with regard to any disclosure of issues relating to stability and/or discoloration.

It would be useful to find formulations of acadesine that have long term stability and are free of discoloration/degradation problems which can be used to treat/prevent ischemic conditions, conditions regulated by adenosine, effects of reduced blood flow to a tissue or simply prevent morbidity/mortality in a patient. The present invention provides such formulations.

### SUMMARY OF THE INVENTION

The present invention provides buffered formulations of acadesine that are stable and substantially free of discoloration.

The present inventors have surprisingly discovered that the presence of oxygen in buffered solutions of acadesine leads to discoloration. Without wishing to be bound by theory, it is believed that the discoloration is due to formation of degradation and/or oxidation product(s) of acadesine. By controlling the level of oxygen, the discoloration in buffered solutions of acadesine can be prevented/minimized.

Three sources of oxygen were identified for vialed product: 1) oxygen dissolved in the water for injection (WFI) (solvent); 2) oxygen in the headspace of the vial; 3) oxygen permeating the rubber stopper closure during shelf life.

Oxygen can be controlled by: 1) headspace minimization; 2) oxygen removal via WFI sparging with nitrogen (before and/or during compounding); 3) vial content overlay with argon (prior to closure); and 4) antioxidant use.

In some embodiments, the present invention provides a buffered solution comprising acadesine (5-Aminoimidazole-4-Carboxamide), wherein said buffered solution has a pH of 6.0-9.6, and a headspace oxygen content of 15% (less than or equal to 15%), and wherein said buffered solution is substantially free of discoloration, said buffered solution being characterized by having less than or equal to (≦) 5 APHA units.

The present invention also provides an acadesine composition comprising an admixture of the aforementioned buffered solution and an aqueous solution distinct from said buffered solution, said aqueous solution selected from the group consisting of normal saline solution, dextrose solution, and cardioplegic solution.

The present invention also provides a kit comprising (1) either (a) a container comprising the aforementioned buffered solution; (b) a container comprising the aforementioned acadesine composition; or (c) a first container comprising the aforementioned buffered solution, and a second container comprising the aforementioned aqueous solution, and (2) instructions for use thereof in treating or preventing a condition in a patient.

The present invention also provides a method of treating a condition in a patient wherein the method comprises the step of administering to the patient an effective amount of the buffered solution or the acadesine composition as set forth above.

All publications and patent applications mentioned in this specification are herein incorporated by reference to the same extent as if each individual publication or patent application was specifically and individually indicated to be incorporated by reference.

### DETAILED DESCRIPTION

The term “patient” is preferably a mammal, and more preferably a human.

The term “treat”, “treating” or “treatment” as used herein refers to inhibiting, retarding ameliorating, or reducing a symptom or a condition affecting an organism.

“Effective amount” or “therapeutically effective amount” is meant to describe an amount of compound or a composition of the present invention effective in inhibiting or treating the conditions or diseases set forth herein and thus producing the desired therapeutic, ameliorative, inhibitory or preventative effect.

The term “co-administered” as used herein refers to administration of two or more agents as part of the same treatment plan, whether or not simultaneous in time or not. Preferably, two agents are co-administered such that their biological activity overlaps in time.

The phrase “substantially free of discoloration” herein means that the solution or composition of the present invention has APHA color units of less than or equal to 5 units (≦5 APHA units). Clear water for example, ideally has zero APHA color units.

The phrase “APHA color units” or “APHA color number” and methods of measuring them are well known to those of ordinary skill in the art, and are described for example in ASTM-D-5386. In the actual measurement, either of a visual comparison with standard samples or a method using a commercially available colorimeter may be employed.

The exact procedure used to determine APHA color number for the present invention is as follows:

### Procedure to Determine APHA Color Number

Instrumentation and Equipment: A spectrophotometer capable of measuring absorbance at 456 nm.

Reagents and materials: Platinum cobolt color standard (APHA No. 500 color standard); Sterile Water for Injection; Deionized water; Curvettes (1-cmpath length)

Standard Solutions: Prepare a series of standard solutions to cover the range of 10 to 500 APHA color units. Add the amounts of platinum cobalt color (APHA 500) standard indicated in the table below to the respective volumetric flasks and dilute each flask to volume with deionized water.

| Standard | Volume of APHA 500 | Volumetric Flask Size | APHA |
|---|---|---|---|
| Solution | (mL) | (mL) | Color units |
| 1 | as is | No Dilution | 500 |
| 2 | 14 | 20 | 350 |
| 3 | 8 | 20 | 200 |
| 4 | 5 | 50 | 50 |
| 5 | 2 | 100 | 10 |

Preparation of the Standard Curve: Transfer a sufficient amount of deionized water to a curvette to within about half of a centimeter from the top. Zero the spectrophotometer at 456 nm. Using the same curvette or a matching curvette, record the absorbance (A_{S}) of each of the five standard solutions at 456 nm. Empty the curvette carefully after each reading and rinse it several times with the next solution to be measured, making sure the outside of the curvette is clean and dry before recording the absorbance. Construct a standard curve: Plot Absorbance (A_{S}) versus APHA color units (C_{S}) for each of the five standard solutions prepared in section C. Determine the slope (m) and y-intercept (b) of the linear regression plot using the following equation:

A_{S}=mC_{S}+b

Calculate the correlation coefficient, R².

A typical standard curve can be represented by a straight line relationship between AU as a function of APHA Color unit, e.g., wherein the line includes the points defined, for example, by AU=0.17 when APHA Color Units are 500, and by AU=0.06 when APHA Color Units are 100.

System Suitability Requirement: The correlation coefficient (R²) of the regression plot of the standard solutions (standard curve, A_{S} versus C_{S}) is equal to or greater than 0.99.

Sample preparation: Allow vial containing test sample to reach room temperature. Wipe the exterior of the vial to remove an external condensation. Remove the plastic top and aluminum seal. Transfer 41.0 mL of Sterile Water for Injection to the vial. Manually shake the vial to dissolve sample. Label: Sample Solution.

Sample Analysis:

Description—Inspect the content of the vial and report the following: a. clarity of the solution, and b. presence of any visible particles.

APHA Color—Transfer a sufficient amount of Sample Solution to a curvette. Measure and record the absorbance (Aᵤ) at 456 nm. Calculate the APHA Color Units (Cᵤ) of the sample using the following equation:

Cᵤ=(Aᵤ−b)/m

### Round the APHA Color Units to a Whole Number and Report the Final Result.

The following table (Table 1) demonstrates that buffered solutions of acadesine without oxygen control (e.g., as measured by headspace oxygen content) are not substantially free of discoloration.

TABLE 1
50 mM TRIS-HCl Buffer, Acadesine 18 mg/mL

| | | | Days at ambient (RT, 21% |
|---|---|---|---|
| Sample | APHA Color^{$} | Appearance | Headspace O₂) |
| pH 6* | 45 | Yellow | 21 |
| pH 7 | 23 | Light yellow | 20 |
| pH 8 | 23 | Light yellow | 250 |
| pH 9 | 13 | Very light yellow | 210 |
| pH 10 | 10 | Very light yellow | 210 |

^{$}Very light yellow color develops at APHA ~10 Units
*At pH 6, very light yellow color was observed in seven days. APHA was not measured.
RT = room temperature
TRIS-HCl buffer is Tris(hydroxymethyl)aminomethane HCl buffer

This table shows that in a buffered acadesine solution (50 mM TRIS-HCl Buffer, Acadesine 18 mg/mL) in ambient conditions (room temperature, 21% headspace oxygen), without oxygen control, discoloration takes place from about seven days (pH 6.0) to about seven months (pH 10).

In one embodiment, the buffer used to prepare the buffered solution of the present invention is selected from the group consisting of: citrate, phosphate, and TRIS-Hydrochloride.

In a preferred embodiment, the buffer utilized is TRIS-Hydrochloride.

In some embodiments, the buffer strength of the buffer utilized in the present invention ranges from 10 mM to 100 mM, and in another embodiment, 50 mM.

In some embodiments, the buffered solution of the present invention further comprises a solubilizer selected from the group consisting of polyethylene glycol and propylene glycol. Table 2 gives further details of some solubilizers utilized in the present invention.

TABLE 2
Solubilizers studied for Acadesine Injectable Solution, 18 mg/mL

| Name | Synonyms | CAS# | Formula | MW | Ref. | Structure |
|---|---|---|---|---|---|---|
| Propylene glycol | propylene glycol, 1,2-propanediol, 1,2-Dihydroxy- propane, methyl ethyl glycol (MEG), methyl- ethylene glycol, PG, Sirlene, Dowfrost | 57-55-6 | C₃H₈O₂ | 76.09 | USP | 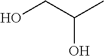 |
| Poly- ethylene glycol | Carbowax, GoLYTELY, GlycoLax, Fortrans, TriLyte, Colyte, Halflytely, Macrogol, MiraLAX, MoviPrep, and PEG-100 Stearate | 25322-68-3 | C₂ₙ₊₂H₄ₙ₊₆Oₙ₊₂ | 44n + 62 | | 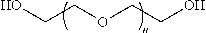 |

In certain embodiments, the buffered solution of the present invention has a pH of 7.2-8.0.

In certain embodiments, the buffered solution of the present invention has a pH of 7.2-8.0, and is further characterized by having ≦0.01 ppm dissolved oxygen and ≦1% headspace oxygen.

In certain embodiments, the buffered solution of the present invention has a pH of 7.2-8.0, and is further characterized by having ≦0.01 ppm dissolved oxygen and ≦1% headspace oxygen, wherein said dissolved oxygen content is achieved by the use of nitrogen gas sparging.

In certain embodiments, the buffered solution of the present invention has a pH of 7.2-8.0, and is further characterized by having ≦0.01 ppm dissolved oxygen and ≦1% headspace oxygen, wherein said dissolved oxygen content is achieved by the use of nitrogen gas sparging.

In certain embodiments, the buffered solution of the present invention has a pH of 7.2-8.0, and is further characterized by having ≦0.01 ppm dissolved oxygen and ≦1% headspace oxygen, when said headspace oxygen content is achieved by the use of nitrogen gas or argon gas overlay, the latter being preferred.

In certain embodiments, the buffered solution of the present invention has a pH of 7.2-8.0, and is further characterized by having ≦0.01 ppm dissolved oxygen and ≦1% headspace oxygen, wherein said buffer solution is terminally sterilizable at 121° C. up to 40 minutes.

In certain embodiments, the buffered solution of the present invention has a pH of 7.2-8.0, has ≦0.01 ppm dissolved oxygen and ≦1% headspace oxygen, and comprises:

a) 1.7-6.0 wt % acadesine;

b) 0.55-0.65 wt % tromethanime (TRIS base);

c) 0.30-0.50 wt % concentrated hydrochloric acid (37% HCl); and

d) 92.8-97.5 wt % of water.

In certain embodiments, the buffered solution of the present invention has a pH of 7.2-8.0, has ≦0.01 ppm dissolved oxygen and ≦1% headspace oxygen, and comprises:

a) 1.79 wt % acadesine;

b) 0.60 wt % tromethanime (TRIS base);

c) 0.32-0.47 wt % concentrated hydrochloric acid (37% HCl); and

d) 97.1-97.3 wt % of water.

In certain embodiments, the buffered solution of the present invention has a pH of 7.2-8.0, has ≦0.01 ppm dissolved oxygen and ≦1% headspace oxygen, and comprises:

a) 5.96 wt % acadesine;

b) 0.60 wt % tromethanime (TRIS base);

c) 0.32-0.47 wt % concentrated hydrochloric acid (37% HCl); and

d) 97.97-93.12 wt % of water.

In certain embodiments, the buffered solution of the present invention has a pH of 7.2-8.0, has ≦0.01 ppm dissolved oxygen and ≦1% headspace oxygen, and wherein the acadesine is present in the buffered solution at a concentration of about 18-60 mg of acadesine per mL of solution.

In certain embodiments, the buffered solution of the present invention has a pH of 7.2-8.0, has ≦0.01 ppm dissolved oxygen and ≦1% headspace oxygen, and wherein the acadesine is present in the buffered solution at a concentration of 18 mg of acadesine per mL of solution.

In certain embodiments, the buffered solution of the present invention has a pH of 7.2-8.0, has ≦0.01 ppm dissolved oxygen and ≦1% headspace oxygen, and wherein the acadesine is present in the buffered solution at a concentration of 60 mg of acadesine per mL of solution.

In certain embodiments, the buffered solution of the present invention has a pH of 6.0-8.0.

In certain embodiments, the buffered solution of the present invention has a pH of 6.0-8.0, and is further characterized by having ≦0.01 ppm dissolved oxygen and ≦3% headspace oxygen.

In certain embodiments, the buffered solution of the present invention has a pH of 6.0-8.0, and is further characterized by having ≦0.01 ppm dissolved oxygen and ≦3% headspace oxygen, wherein said dissolved oxygen content is achieved by the use of nitrogen gas sparging.

In certain embodiments, the buffered solution of the present invention has a pH of 6.0-8.0, and is further characterized by having ≦0.01 ppm dissolved oxygen and ≦3% headspace oxygen, when said headspace oxygen content is achieved by the use of nitrogen gas overlay or argon gas overlay, the latter being preferred.

In certain embodiments, the buffered solution of the present invention has a pH of 6.0-8.0, and is further characterized by having ≦0.01 ppm dissolved oxygen and ≦3% headspace oxygen, and is terminally sterilizable at 121° C. up to 40 minutes.

In certain embodiments, the buffered solution of the present invention has a pH of 6.0-8.0, and is further characterized by having ≦0.01 ppm dissolved oxygen and ≦3% headspace oxygen, and further comprises an antioxidant.

In certain embodiments, the antioxidant of the present invention is selected from the group consisting of sodium sulfite, sodium hydroxymethanesulfinate, sodium bisulfite, sodium metabisulfite, and acetone sodium bisulfite.

Table 3 lists several antioxidants utilized in the formulation of the buffered solutions of the present invention.

TABLE 3
Antioxidants studied for Acadesine Injectable solution, 18 mg/mL

| Name | Synonyms | CAS # | Formula | Structure | Mw | Ref |
|---|---|---|---|---|---|---|
| Acetone Sodium Bisulfite | 2-Hydroxy-2- propanesulfonic acid sodium salt | 540-92-1 | C₃H₇NaO₄S | 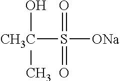 | 162.14 | |
| Sodium Hydroxymethanesulfinate hydrate | Hydroxymethanesulfinic acid sodium salt, Rongalit ™, Sodium formaldehyde sulfoxylate hydrate, Sodium hydroxymethanesulfinate hydrate | 149-44-0 | CH₃SO₃Na | 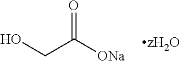 | 118.09 (anhydr. basis) | |
| Thioglycerol | α-Monothioglycerol, α-Thioglycerol, 3- Mercapto-1,2- propanediol | 96-27-5 | C₃H₈SO₂ | 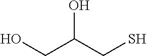 | 108.16 | |
| Gentisic Acid Sodium Salt (hydrate) | 2,5-dihydroxybenzoic acid sodium salt | 4955-90-2 | C₇H₅O₄Na | 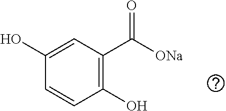 | 176.10 | |
| Cysteine Hydrochloride Monohydrate | 2-amino-3-sulfanyl- propanoic acid hydrochloride | 7048-04-6 | C₃H₈ClNO₂S | 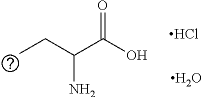 | 175.63 | USP EP |
| Sodium Sulfite (anhydrous) | | 7757-83-7 | Na₂SO₃ | Na₂SO₃ | 126.04 | EP |
| Sodium Bisulfite | | 7631-90-5 | NaHSO₃ | NaHSO₃ | 104.0 | |
| Sodium Metabulfite | Sodium disulfite, Sodium pyrosullite | 7681-57-4 | Na₂S₂O₅ | Na₂S₂O₅ | 190.11 | NF BP EP |
| Sodium Ascorbate | L(+)-Ascorbic acid sodium salt, Vitamin C sodium salt | 134-03-02 | C₆H₇NaO₆ | 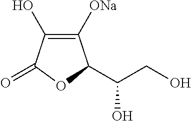 | 198.11 | |

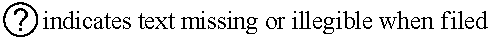

In certain embodiments, the buffered solution of the present invention has a pH of 6.0-8.0, and is further characterized by having ≦0.01 ppm dissolved oxygen and ≦3% headspace oxygen, wherein the buffered solution comprises:

a) 1.7-6.0 wt % acadesine;

b) 0.55-0.65 wt % tromethanime (TRIS base);

c) 0.32-0.52 wt % concentrated hydrochloric acid (37% HCl);

d) 0.07-0.11% anhydrous sodium sulfite; and

d) 92.8-97.2 wt % of water.

In certain embodiments, the buffered solution of the present invention has a pH of 6.0-8.0, and is further characterized by having ≦0.01 ppm dissolved oxygen and ≦3% headspace oxygen, wherein the buffered solution comprises:

a) 1.79 wt % acadesine;

b) 0.60 wt % tromethanime (TRIS base);

c) 0.32-0.52 wt % concentrated hydrochloric acid (37% HCl);

d) 0.07-0.11% anhydrous sodium sulfite; and

d) 97.02-97.18 wt % of water.

In certain embodiments, the buffered solution of the present invention has a pH of 6.0-8.0, and is further characterized by having 0.01 ppm dissolved oxygen and 3% headspace oxygen, wherein the buffered solution comprises:

a) 5.96 wt % acadesine;

b) 0.60 wt % tromethanime (TRIS base);

c) 0.32-0.52 wt % concentrated hydrochloric acid (37% HCl);

d) 0.07-0.11% anhydrous sodium sulfite; and

d) 92.85-93.01 wt % of water.

In certain embodiments, the buffered solution of the present invention has a pH of 6.0-8.0, and is further characterized by having ≦0.01 ppm dissolved oxygen and ≦3% headspace oxygen, and wherein the buffered solution has an acadesine concentration of about 18-60 mg of acadesine per mL of solution.

In certain embodiments, the buffered solution of the present invention has a pH of 6.0-8.0, and is further characterized by having ≦0.01 ppm dissolved oxygen and ≦3% headspace oxygen, and wherein the buffered solution has an acadesine concentration of about 18 mg of acadesine per mL of solution.

In certain embodiments, the buffered solution of the present invention has a pH of 6.0-8.0, and is further characterized by having ≦0.01 ppm dissolved oxygen and ≦3% headspace oxygen, and wherein the buffered solution has an acadesine concentration of about 60 mg of acadesine per mL of solution.

In another embodiment, the buffered solution of the present invention has a pH of 8.8-9.6.

In another embodiment, the buffered solution of the present invention has a pH of 8.8-9.6, and is characterized by having ≦0.01 ppm dissolved oxygen and ≦15% headspace oxygen.

In another embodiment, the buffered solution of the present invention has a pH of 8.8-9.6, and is characterized by having ≦0.01 ppm dissolved oxygen and ≦15% headspace oxygen, wherein said dissolved oxygen content is achieved by the use of nitrogen gas sparging.

In another embodiment, the buffered solution of the present invention has a pH of 8.8-9.6, and is characterized by having ≦0.01 ppm dissolved oxygen and ≦15% headspace oxygen, wherein said headspace oxygen content is achieved by the use of nitrogen gas or argon gas overlay, the latter being preferred.

In another embodiment, the buffered solution of the present invention has a pH of 8.8-9.6, and is characterized by having ≦0.01 ppm dissolved oxygen and ≦15% headspace oxygen, wherein said solution is terminally sterilizable at 121° C. up to 40 minutes.

The tables 4-6 below illustrate the relative amounts of each component of the present buffered solution comprising acadesine at different pH ranges:

TABLE 4
Formulation for Acadesine Injectable Solution
18 mg/mL - 60 mg/mL^{#}, pH 6.0 - 8.0, target pH 7.0 (with antioxidant)

| Components | Function | % by weight |
|---|---|---|
| Acadesine | Active | 1.79^{$} |
| | | 5.96^{&} |
| Tromethamine (TRIS | Buffering agent | 0.60 |
| base) | | |
| Hydrochloric Acid | Buffering agent | 0.32-0.52 |
| Sodium Sulfite, | Antioxidant | 0.07-0.11 |
| Anhydrous* | | |
| Water for Injection** | Solvent | 97.02-97.18^{$} |
| | | 92.85-93.01^{&} |

^{#}5° C. solubility data supports 18 mg/mL; Room temperature solubility data supports 60 mg/mL
*Can be excluded from formulation if there is sufficient oxygen control
**Nitrogen gas is used for WFI sparging (0.01 ppm dissolved Oxygen) and Argon gas is used for overlay (≦3% headspace oxygen) to prevent oxidation and discoloration of formulated product
^{$}Acadesine concentration @ 18 mg/mL
^{&}Acadesine concentration @ 60 mg/mL

TABLE 5
Formulation for Acadesine Injectable Solution
18 mg/mL - 60 mg/mL^{#}, pH 7.2 - 8.0, target pH 7.6

| Components | Function | % by weight |
|---|---|---|
| Acadesine | Active | 1.79^{$} |
| | | 5.96^{&} |
| Tromethamine | Buffering | 0.60 |
| (TRIS base) | agent | |
| Hydrochloric Acid | Buffering | 0.32-0.47 |
| | agent | |
| Water for Injection* | Solvent | 97.14-97.29^{$} |
| | | 92.97-93.12^{&} |

^{#}5° C. solubility data supports 18 mg/mL; Room temperature solubility data supports 60 mg/mL
*Nitrogen gas is used for WFI sparging (0.01 ppm dissolved Oxygen) and Argon gas is used for overlay (≦1% headspace oxygen) to prevent oxidation and discoloration of formulated product
^{$}Acadesine concentration @ 18 mg/mL
^{&}Acadesine concentration @ 60 mg/mL

TABLE 6
Formulation for Acadesine Injectable Solution
mg/mL - 60 mg/mL^{#}, pH 8.8 - 9.6, target pH 9.2

| Components | Function | % by weight |
|---|---|---|
| Acadesine | Active | 1.79$ |
| | | 5.96& |
| Tromethamine | Buffering agent | 0.60 |
| (TRIS base) | | |
| Hydrochloric Acid | Buffering agent | 0.02-0.10 |
| Water for Injection** | Solvent | 97.51-97.59$ |
| | | 93.33-93.41& |

**Nitrogen gas is used for WFI sparging (0.01 ppm dissolved Oxygen) and for overlay (≦15% headspace oxygen) to prevent oxidation and discoloration of formulated product
^{#}50 C. solubility data supports 18 mg/mL; Room temperature solubility data supports 60 mg/mL
^{$}Acadesine concentration @ 18 mg/mL
^{&}Acadesine concentration @ 60 mg/mL

In certain embodiments, the present invention provides an acadesine composition comprising an admixture of the buffered solution of the present invention and an aqueous solution distinct from said buffered solution, said aqueous solution selected from the group consisting of normal saline solution, dextrose solution, and cardioplegic solution.

In certain embodiments, the dextrose solution is selected from the group consisting of a) 2.5% dextrose solution in water (D2.5W), and b) 5% dextrose solution in water (D5W).

In certain embodiments, the cardioplegic solution is selected from the group consisting of reperfusate, induction, ringers, and maintenance solutions.

In general there are four standard cardioplegic bags made. They are reperfusate, induction, ringers, and maintenance. Isolyte-S is a multi-electrolyte injection. Each 100 mL of Isolyte S contains Sodium Chloride USP 0.53 g; Sodium Gluconate USP 0.5 g; Sodium Acetate Trihydrate USP 0.37 g; Potassium Chloride USP 0.037 g; Magnesium Chloride Hexahydrate USP 0.03 g; and Water for Injection USP qs.

### Reperfusate

### Mannitol 20% 37.5 mL

### Isolyte-S pH 7.4 291.75 mL

Citrate-Phosphate-Dextrose (CPD) 30 mL

monosodium L-glutamate (MSA)/monosodium L-aspartate (MSG) 0.92M 70 mL Add prior to use: Sodium bicarbonate 62.5 mEq (62.5 mL); Lidocaine 2% 125 mg (6.2 mL); and Nitroglycerin 1000 mcg (0.2 mL)

### Induction

### Sodium Bicarbonate 8.4% 31.25 mEq

### Potassium Chloride 35 mEq

### Mannitol 25% 3.75 g

### Isolyte-S pH 7.4 133 mL

Add prior to use with induction: Lidocaine 2% 62.5 mg; Nitroglycerin 500 mcg; and

### Albumin 25% 12.5 g

### Ringers

### Ringer's Solution 1000 mL

### Potassium Chloride 20 mEq

### Magnesium Chloride 32 mEq

### Mannitol 20% 10 g

### Sodium Bicarbonate 8.4% 6.5 mEq

Add prior to use: Procaine 10% 2.73 mL

### Maintenance

### Sodium Bicarbonate 8.4% 125 mEq

### Potassium Chloride 25 mEq

### Mannitol 25% 15 g

### Isolyte-S pH 7.4 802 mL

In certain embodiments, the acadesine composition of the present invention comprises an admixture of the buffered solution of the present invention and a normal saline solution, wherein the concentration of acadesine in said admixture ranges from 3.3-12.6 mg/mL.

In certain embodiments, the acadesine composition of the present invention comprises an admixture of the buffered solution of the present invention and a normal saline solution, wherein the concentration of acadesine in said admixture ranges from 3.3-12.6 mg/mL, and wherein the difference between the pH of the admixture and the pH of the buffered solution is ≦0.1.

In certain embodiments, the acadesine composition of the present invention comprises an admixture of the buffered solution of the present invention and a normal saline solution, wherein the concentration of acadesine in said admixture ranges from 3.3-12.6 mg/mL, wherein the difference between the pH of the admixture and the pH of the buffered solution is ≦0.1, and wherein the acadesine composition has an Osmolality of 183-257 mOsm/Kg.

As per clinical practice, a solution containing 1 mole (1 gram molecular weight) of a non-ionizable substance in 1 kg of water (a 1 molal solution) is referred to as a 1 osmolal solution. It contains 1 osmol or 1000 milliosmols (mOsm) of solute per kilogram of solvent. Osmolality measures the total number of particles dissolve in a kilogram of water, that is, the osmols per kilogram of water, and depends on the electrolyte nature of solute.

In blood and other body fluids, osmolality is mainly contributed by sodium chloride. The osmolality of blood obtained by various workers using different experimental techniques, ranges from 250 to 350 mOsm/kg.

Table 7 below illustrates the relative volumes of acadesine (at 18 mg/mL) and 0.9% NaCl (mimicking normal saline solution) in the acadesine composition need to make up a final volume of 25 mL of acadesine composition at final acadesine concentrations of 3.36 mg/mL and 12.60 mg/mL.

Table 8 illustrates the pre-admixture pH, post-admixture pH, and the Osmolality of the acadesine compositions at various conditions.

TABLE 7

| | Acadesine | | |
|---|---|---|---|
| | 18 mg/mL (mL) | 0.9% NaCl (mL) | |
| | (from each of 11 | (from PVC-DEHP IV | |
| | batches | bags - Viaflex | Total |
| | 63168-103-X | container, | Volume |
| Dose | stored @ 5° C.) | Baxter 2B1322) | (mL) |
| Low, 3.36 mg/mL | 4.65 | 20.35 | 25 |
| High, 12.60 mg/mL | 17.5 | 7.5 | 25 |

TABLE 8

| | | pH | Osmolality |
|---|---|---|---|
| | | post-admixture | mOsm/Kg |
| | pH | Low Dose pH | Low Dose pH |
| Batch | pre-admixture | High Dose pH | High Dose pH |
| Acadesine 18 mg/mL | 8.1 | 8.1 | 274 |
| 100 mM Tris-HCl | | 8.1 | 239 |
| Buffer, pH 8.0 | | | |
| Acadesine 18 mg/mL | 8.0 | 8.0 | 257 |
| 50 mM Tris-HCl | | 8.0 | 183 |
| Buffer, pH 8.0 | | | |
| Acadesine 18 mg/mL | 8.1 | 8.1 | 251 |
| 25 mM Tris-HCl | | 8.0 | 158 |
| Buffer, pH 8.0 | | | |
| Acadesine 18 mg/mL | 8.0 | 8.0 | 246 |
| 10 mM Tris-HCl | | 8.0 | 143 |
| Buffer, pH 8.0 | | | |
| Acadesine 18 mg/mL | 7.2 | 6.5 | NT |
| No Buffer, pH 8.0 | | 6.7 | NT |
| with NaOH | | | |
| Acadesine 18 mg/mL | 9.1 | 9.1 | NT |
| 100 mM Tris-HCl | | 9.1 | NT |
| Buffer, pH 9.0 | | | |
| Acadesine 18 mg/mL | 9.0 | 9.1 | NT |
| 50 mM Tris-HCl | | 9.1 | NT |
| Buffer, pH 9.0 | | | |
| Acadesine 18 mg/mL | 9.0 | 9.0 | NT |
| 25 mM Tris-HCl | | 9.1 | NT |
| Buffer, pH 9.0 | | | |
| Acadesine 18 mg/mL | 9.0 | 8.9 | NT |
| 10 mM Tris-HCl | | 9.0 | NT |
| Buffer, pH 9.0 | | | |
| Acadesine 18 mg/mL | 7.3 | 6.5 | NT |
| No Buffer, pH 9.0 | | 6.6 | NT |
| with NaOH | | | |
| Acadesine 18 mg/mL | 6.8 | 6.4 | NT |
| No Buffer, No pH | | 6.6 | NT |
| adjustment | | | |
| Control | 6.2 | N/A | NT |

The present invention also provides a method of treating a condition in a patient wherein the method comprises the step of administering to the patient an effective amount of the buffered solution of the present invention or the acadesine composition of the present invention described above.

Examples of conditions treatable or preventable by the solutions or compositions herein include an ischemic condition, a condition regulated by adenosine, or a condition associated with reduced blood flow to a tissue. In preferred embodiments, the condition being treated is a myocardial infarction, stroke, or death.

Examples of condition that can be treated by the compositions and solutions herein include those selected from the group consisting of a heart attack, a stroke, death (e.g., sudden cardiac death), a myocardial infarction (e.g., a transmural or non-transmural myocardial infarction or an acute myocardial infarction), coronary artery disease, coronary heart disease, an arrhythmia, a cerebrovascular accident, congestive heart failure, a life-threatening dysrhythmia, cardiomyopathy, an ischemic condition (e.g., a transient ischemic attack, an acute ischemic syndrome, acute bowel ischemia, kidney ischemia), angina pectoralis, a vascular condition (e.g., microvascular disease of diabetes mellitus, disseminated intravascular coagulation such as due to bowel ischemia), intermittent claudication of skeletal muscle, migraine headaches, Raynauds phenomenon, a hepatic injury, a pancreatic injury, shock, or a combination thereof. In some embodiments, the condition is death, a myocardial infarction or a heart attack.

In certain embodiments, the patient being administered a composition/solution of the present invention is undergoing surgery. In such embodiment, the compositions/solutions herein can be administered perioperatively. In some embodiments, the patient is undergoing a cardiac surgery; while in other embodiments, the patient is undergoing a non-cardiac surgery.

Examples of cardiac surgeries include those selected from the group consisting of coronary artery bypass grafting (CABG), percutaneous transluminal coronary angioplasty (PTCA), percutaneous transluminal angioplasty (PTA) laser angioplasty, cerebral angioplasty, an atherectomy, an intravascular stent procedure, carotid endarterectomy, heart and heart-lung transplant, implantation of artificial heart devices and defibrillators, valve replacement or repair, and congenital surgery. In preferred embodiments, the compositions/solutions herein are administered to a patient undergoing a CABG surgery.

In certain embodiments, a cardiac surgery is an intravascular stent procedure. An intravascular stent is a device adapted to be implanted into the blood vessel or coronary artery of a patient. Generally, stents are cylindrical devices capable of holding open and sometimes expanding a segment of a blood vessel or a coronary artery. Stents may be delivered in a compressed form to a target location and then deployed in an expanded form to support the vessel or artery and prevent a reclosure. Stents may be used to treat atherosclerotic stenosis in an artery and/or blood vessel or to treat and repair blood vessels following a narrowing or stenosis in the artery or blood vessel.

Preferably under the present invention, the cardiac surgery may involve the use of a stent to treat and/or repair blood vessels after a stenosis has been compressed by a PTCA or a PTA, or after a stenosis has been removed by an atherectomy by improving the result of the procedure and/or reducing the possibility of a reclosure or restenosis.

The cardiac surgery may also involve the use of a stent to compress a stenosis without an initial procedure such as a PTCA or a PTA. Under the present invention, the surgery may involve the implantation of a stent on another body lumen such as carotid arteries, peripheral vessels, urethra, esophagus and bile duct. The cardiac surgery related to a vascular repair device may be an angioplasty procedure.

In one embodiment, the compositions/solutions of the present invention may be used in a drug-coated or drug-eluting stent where the outer portion of a standard intravascular stent is coated with a drug. Preferably, a polymer containing a composition described herein or a formulation thereof may be used to coat an intravascular stent.

Following deployment of the stent, the compositions herein may diffuse out into the wall of the vessel or artery over the time following cardiac surgery to reduce and/or prevent a condition, which can be treated and/or prevented by increasing the endogenous localized level of extracellular adenosine.

Examples of non-cardiac surgeries include those selected from the group consisting of non-heart organ transplant, small and large bowel resection, appendectomy, laparoscopy, paracentesis, transurethral resection of the prostate (TURP), hysterectomy, tubal ligation, vasectomy, salpingo-oophorectomy, Cesarean section, hemorrhoidectomy, tonsillectomy, myringodectomy, placement of myringotomy tubes, removal of polyp(s) from the colon and rectum, repair of rectal prolapse, removal and treatment of neoplasms of the bowel, curettage, thoracentesis, thoracotomy, rhinoplasty, and liposuction, or a combination thereof.

The compositions/solutions herein are preferably administered vie intravenous (i.v.) injection or via a perfusate solution in an effective amount.

An effective amount of the buffered solution of the present invention herein include an amount that is between 0.001 mg/kg/mm to 20 mg/kg/mm, or more preferably about 0.1 mg/kg/mm. At such rates, the solution herein can be administered for at least 1 hour, or more preferably for about 7 hours.

The methods herein can treat any of the conditions herein, but the solutions and compositions of the present invention can also be used to prevent death, stroke, or a myocardial infarction in a patient undergoing CABG surgery.

The present invention also provides a kit comprising (1) either (a) a container comprising the aforementioned buffered solution; (b) a container comprising the aforementioned acadesine composition; or (c) a first container comprising the aforementioned buffered solution, and a second container comprising the aforementioned aqueous solution, and (2) instructions for use thereof in treating or preventing a condition in a patient.

In some embodiments, the invention relates to a perfusate solution. Such perfusate solution comprises, consists essentially of, or consists of the buffered solution or the acadesine composition described above.

The compositions/solutions (including perfusate solution) herein can include a second therapeutic agent.

Examples of a second therapeutic agent include, but are not limited to an adenosine deaminase inhibitor, a blood-clotting inhibitor, an anticoagulant, an anti-platelet agent, an anti-hypertensive agent, a cholesterol-lowering drug, a vasodilator, a beta-blocker, an ace-inhibitor, an analgesic, an anti-inflammatory agent, and a diuretic. In some embodiments, the second therapeutic agent is pentostatin.

In any of the embodiments herein, the buffered solution or the acadesine composition of the present invention can be formulated for slow release and/or for administration via a drug eluting stent.

In any of the formulations herein, one or more of the buffered solution or the acadesine composition may be present in an “effective amount”, i.e., in an amount effective to achieve therapeutic and/or prophylactic benefit.

Pharmaceutical formulations herein can optionally include one or more pharmaceutically acceptable carriers, excipients or diluents.

The buffered solution of the present invention is preferably provided in a concentrated form suitable for dilution. The solution can then be further diluted by a health care provider prior to administration to a patient. In preferred embodiments, the concentrated solution above is diluted in normal saline prior to administration. Dilutions are performed by health care provider based on amount of acadesine to be administered over the course of treatment.

In certain embodiments, the buffered solutions, compositions, or formulations herein are administered perioperatively. For example, an i.v. solution comprising, consisting essentially of, or consisting of a composition herein can be administered beginning between 1-90 minutes before anesthesia, 2-80 minutes before anesthesia, 3-70 minutes before anesthesia, 4-60 minutes before anesthesia, 5-50 minutes before anesthesia, 6-40 minutes before anesthesia, 7-30 minutes before anesthesia, 8-28 minutes before anesthesia, 9-26 minutes before anesthesia, 10-24 minutes before anesthesia, 11-22 minutes before anesthesia, 12-20 minutes before anesthesia, 13-18 minutes before anesthesia, 14-16 minutes before anesthesia, or preferably 15 minutes before anesthesia.

The buffered solutions, formulations and/or compositions herein can be administered for a period of 1 to 24 hours, 2 to 20 hours, 3 to 16 hours, 4 to 12 hours, 5 to 10 hours, 6 to 8 hours, or preferably 7 hours.

In certain embodiment, the buffered solutions, formulations and/or compositions herein are administered post-surgery for a period of between 1 to 36 hours, 2 to 32 hours, 3 to 28 hours, 4 to 24 hours, 5 to 20 hours, 6 to 18 hours, 7 to 16 hours, 8 to 14 hours, or 9 to 12 hours.

In certain embodiments, the buffered solutions, formulations and/or compositions herein are administered beginning 15 minutes prior to administration of anesthesia for cardiac surgery (e.g. CABG), and continuing for 7 hours.

However, the rate and time of dosage may be altered depending on a number of variables, not limited to the activity of the composition used, the condition to be treated, the mode of administration, the requirements of the individual subject, the severity of the condition being treated, and the judgment of the practitioner.

The buffered solutions or compositions herein may be co-administered with one or more additional therapeutic agents. The choice of therapeutic agents that can be co-administered with the compositions of the invention will depend, in part, on the condition being treated or prevented.

In certain embodiments, a buffered solution or composition herein is co-administered with an adenosine deaminase inhibitor, a blood-clotting inhibitor, an anticoagulant, an anti-platelet agent, an anti-hypertensive agent, a cholesterol lowering drug, a vasodilator, a beta-blocker, an ace-inhibitor, an analgesic, an anti-inflammatory agent, an anti-neoplastic agent, and/or a diuretic.

In certain embodiments, a buffered solution or composition herein is co-administered with an adenosine deaminase inhibitor. Such an inhibitor can prevent an adenosine deaminase from catalyzing the deamination of adenosine to inosine. (See Law, U.S. Pat. No. 6,103,702).

Examples of adenosine deaminase inhibitors that may be combined with a compositions herein include but are not limited to 9-(1-hydroxy-2-octyl) adenine, erythro-9-(2-hydroxy-3-nonyl)adenine (EHNA), 2′-deoxycoformycin, coformycin, i,3,7-trimethylxanthine (caffeine), and pentostatin. In a preferred embodiment, the present invention contemplates the co-administration or co-formulation of a composition as described herein with pentostatin. More preferably, the present invention relates to a composition or formulation thereof comprising a composition comprising AICA riboside, including compounds of formula I, II, and III or an analog thereof and pentostatin.

In one aspect of the present invention, the buffered solutions or compositions described herein may be co-administered with a blood clotting inhibitor. The blood clotting inhibitor of the present invention can be any drug, agent or pharmaceutical composition that prevents or inhibits blood clotting. The inhibitor can act by preventing or inhibiting blood clot formation by any of a variety of mechanisms including reduction of blood clotting factors or reducing platelet activation or aggregation, or mitigating the effects of instigating factors, such as inflammation or stress. The blood clotting inhibitor can also act by breaking down or dissolving a blood clot after formation. There are several classes of blood clotting inhibitor, including anti-platelet agents, thrombolytic enzymes, aggregation inhibitors, glycoprotein IIb/IIIa inhibitors, glycosaminoglycans, thrombin inhibitors, anticoagulants, heparins, low molecular weight heparins, coumarins, indandione derivatives and tissue plasminogen activators. See, The Physicians' Desk Reference (56th ed., 2002) Medical Economics; Mosby s Drug Consult, 2002, Elsevier Science; Goodman and Gilman's The Pharmacologic Basis of Therapeutics, (9th ed. 1996) Pergamon Press; Drug Facts and Comparisons, updated monthly, September, 2002, Facts and Comparisons, Wolters Kiuwer Company, St. Louis, Mo.

For the purposes of this invention, any substance that prevents or inhibits the formation of blood clots or dissolves or breaks down a blood clot is suitable. Such a blood clotting inhibitor can be, for example, cilostazol (PLETAL®, Otsuka), clopidogrel (PLAVIX®, Sanofi-Aventis), ticlopidine (TICLID®, Syntex), tirofiban (AGGRASTAT®, Medicure International), eptifibatide (INTEGRILIN®, Millennium Pharmaceuticals), abciximab (REOPRO®, Eli Lilly), anagrelide (AGRYLrN®, Roberts), dipyridamole (PERSANTE), Boehringer Ingelheim), aspirin (ECOTR®, and others), dipyridamole/aspirin (AGGRENOX®, Boehringer Ingelheim), dalteparin (FRAGMIN®, Pharmacia), enoxaparin (LOVENOX®, Aventis), tinzaparin (INNOHE®, DuPont), heparin (various), danaparoid (ORGANON®, Organon), antithrombin III (THROMBATE®, Bayer), lepirudin (REFLUDAN®, Sanofi-Aventis), argatroban (ACOVA®, Glaxo SmithKline), bivalirudin (ANGIOMAX®, Medicines Company), warfarin (COUMAD1Nφ, DuPont) anisidione (MIRAD ON®, Schering), alteplase (ACTIVASE®, Genetech), reteplase (RETAVASE®, Boehringer Mamiheim), tenecteplase (TNXASE®, Genentech), drotrecogin (XIGRIS®, Eli Lilly), anistreplase (EMINASE®, Roberts), streptokinase (STREPTASE®, Astra), urokinase (ABBOKINASE®, Abbott) and combinations thereof.

In certain embodiments, the buffered solutions or composition(s) herein are co-administered with a blood clotting inhibitor. Preferably, such blood clotting inhibitor is aspirin.

In certain embodiments, the buffered solution or composition herein are co-administered with an anti-neoplastic agent. Examples of anti-neoplastic agents include, but are not limited to, Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsaniitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil 1131; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Imofosine; Interferon Alfa-2a; Interferon Alfa-2b; Interferon Alfa-ni; Interferon Alfa-n3; Interferon Beta-Ia; Interferon Gamma-Tb; Iproplatin; Irinotecan Hydrochloride; Lanreotide Acetate; Letrozole; Leuprolide Acetate Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Methredepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin;-9-Prfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycinl, Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfm; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfm; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

In one embodiment, the compositions and formulations herein can be used to modulate or increase the level of local endogenous adenosine. Adenosine (Ad) may bind one or more of its known receptors (Adr) including A1, A2A, A2B, and A3, which may modulate various physiological responses that affect conditions, such as e.g. ischemic conditions. For example, adenylate cyclase may be activated where a stimulatory G protein binds an adenosine receptor, thereby leading to cAMP production and the activation of kinases ultimately resulting in vasodilation. Adenosine binding may also inhibit the physiological response where an inhibitory G protein binds an adenosine receptor, thereby leading to an inhibition of adenylate cyclase.

The buffered solutions and compositions of the present invention may be administered to a patient to treat and/or prevent an adenosine receptor-related condition. An adenosine receptor-related condition is one where the activity of an adenosine receptor is implicated. Specifically, the condition may be treated by promoting the binding of adenosine to its receptor thereby increasing the activity of adenylate cyclase or a kinase activated due to the increased activity of adenylate cyclase. The increased activity of adenylate cyclase or one of its substrates may lead to increased vasodilation. An adenosine receptor-related condition may also be treated and/or prevented by inhibiting the binding of adenosine to its receptor. Preferably, the present invention provides a method for treating and/or preventing a condition in a patient comprising administering a composition or formulation thereof described herein wherein the condition is an adenosine receptor-related condition. An adenosine receptor-related condition is one in which the activity of an adenosine receptor is implicated, either through inhibition or through activation of the receptor.

In certain embodiments, the condition may be an adenosine receptor-related condition. Such conditions include, but are not limited to, asthma, allergies, allergic diseases (e.g. allergic rhinitis and -sinusitis), autoimmune diseases (e.g. lupus), diarrheal diseases, insulin resistance, diabetes, prevention of mast cell degranulation associated with ischemia/reperfusion injuries, heart attack, inflammatory condition, thrombotic condition (e.g., pulmonary embolism, acute thrombosis of the coronary arteries, myocardial infarction, acute thrombosis of the cerebral arteries (stroke) or other organs), inhibition of angiogenesis in neoplastic tissues, and inhibition of angiogenesis in diabetic retinopathy or hyperbaric oxygen-induced retinopathy.

The methods of treating a condition described herein involve administering a composition or formulation of the present invention to a patient in an effective amount.

In certain embodiments, an effective amount may be such that it maintains the blood plasma concentration of a composition of the invention from greater than about 0.01 μg/mL to less than about 50 μg/mL, greater than about 0.1 μg/mL to less than about 45 μg/mL, greater than about 1 μg/mL to less than about 35 μg/mL, greater than about 2 μg/mL to less than about 30 μg/mL, greater than about 3 μg/mL to less than about 25 μg/mL, greater than about μg/mL to less than about μg/mL, greater than about 5 μg/mL to less than about 15 μg/mL, greater than about 6 μg/mL to less than about 10 μg/mL, greater than about 7 μg/mL to less than about 9 μg/mL, preferably greater than about 1 μg/mL to less than about 20 μg/mL, more preferably greater than about 3 μg/mL to less than about 6 μg/mL, and most preferably about 5 μg/mL. Under one embodiment, the desired blood plasma concentration of AICA riboside in a patient is achieved after administration within about 1 minute to about 15 minutes, about 2 minutes to about 12 minutes, about 3 minutes to about 10 minutes, about 4 minutes to about 8 minutes, about 5 minutes to about 7, preferably about 2 minutes to about 5 minutes.

In certain embodiments, an effective amount of a buffered solution of composition of this invention herein is 0.001 mg/kg/minute to 20 mg/kg/minute, 0.005 mg/kg/minute to 10 mg/kg/minute, 0.01 mg/kg/minute to 5 mg/kg/minute, 0.05 mg/kg/minute to 1 mg/kg/minute, 0.1 mg/kg/minute to 0.5 mg/kg/minute, and more preferably about 0.1 mg/kg/minute.

In certain embodiments, the present invention provides a method of treating a condition described herein by administering a buffered solution or a composition of the present invention at a dose of 1 mg/kg to 500 mg/kg, 2 mg/kg to 400 mg/kg, 3 mg/kg to 300 mg/kg, 4 mg/kg to 250 mg/kg, 5 mg/kg to 225 mg/kg, 10 mg/kg to 200 mg/kg, 30 mg/kg to 160 mg/kg. In some embodiments, the total dosage is about 40 mg/kg. When administered to a patient undergoing a non-cardiac surgery the total dose maybe at about 100-240 mg/kg.

In any of the embodiments herein, the compositions or formulations may be administered for a period of greater than about 1 minute to less than about a year. In preferred embodiments, such compositions or formulations thereof are administered for a period of time greater than about 1 hour to less than about 1 week, a period of time greater than about 2 hours to less than about 1 day, a period of time greater than about 3 hours to a less than about 18 hours, a period of time greater than about 4 hours to less than about 12 hours, a period of time greater than about 6 hours to less than about 10 hours, and more preferably for a period of time greater than about 4 hours to less than about 8 hours.

Lower or higher doses than those disclosed herein may be used, as required. Such dosages, however, may be altered depending on a number of variables, not limited to the activity of the compound used, the condition to be treated, the mode of administration, the requirements of the individual subject, the severity of the condition being treated, and the judgment of the practitioner. The foregoing ranges are merely suggestive, as the number of variables in regard to an individual treatment regime is large, and considerable excursions from these recommended values are not uncommon.

### EXAMPLES

The following formulations within the scope of the present invention provide illustrations of various buffered solutions of acadesine at various pH ranges.

### Example 1

The first formulation (Table 9) is comprised of 50 mM TRIS-Hydrochloride buffer with pH range of 6.0-8.0 (target pH 7.0), 18 mg/mL of acadesine API and sodium sulfite or sodium hydroxymethansulfinate as antioxidant. Nitrogen sparging (dissolved oxygen ≦0.01 ppm) and overlay with argon gas (headspace oxygen ≦3%) are utilized to provide shelf-life of 2-3 years for acadesine injectable solution, 18 mg/mL.

TABLE 9
Acadesine Injectable Solution, 18 mg/mL, pH 6.0-8.0, target pH 7.0 (with
antioxidant)

| Ingredients of Acadesine | | Concentration |
|---|---|---|
| Solution | Function | (mg/mL) |
| Acadesine (API) | Active | 18.00 |
| Tromethamine (TRIS base) | Buffering agent | 6.06 |
| Hydrochloric Acid | Buffering agent | 4.80 |
| Anhydrous Sodium Sulfite* | Antioxidant | 0.7-1.10 |
| Water for Injection** | Solvent | q.s. to 1.0 mL |

*To control residual oxygen in headspace
**Nitrogen gas is used for WFI sparging (0.01 ppm dissolved Oxygen) and Argon gas is used for overlay (≦3% headspace Oxygen) to prevent oxidation and discoloration of formulated product

Sodium sulfite is used as measure to control discoloration of acadesine injectable solution, 18 mg/mL. The level of antioxidant is 1 mg/mL for oxygen headspace level of 3.0%. This level of antioxidant is driven by headspace in the vials and headspace oxygen content upon closure.

### Example 2

The second formulation (Table 10) is comprised of 50 mM TRIS-Hydrochloride buffer with pH range of 7.2-8.0 (target pH 7.6). Concentration of API (acadesine) is 18 mg/mL. Nitrogen sparging (dissolved oxygen ≦13.01 ppm) and overlay with argon gas (headspace oxygen <1%, before vial closure) are utilized to provide shelf-life of 2-3 years for acadesine injectable solution, 18 mg/mL.

TABLE 10
Acadesine Injectable Solution
18 mg/mL, pH 7.2 - 8.0, target pH 7.6

| | | Concentration |
|---|---|---|
| Ingredients of Acadesine Solution | Function | (mg/mL) |
| Acadesine (API) | Active | 18.00 |
| Tromethamine (TRIS base) | Buffering agent | 6.06 |
| Hydrochloric Acid | Buffering agent | 4.12 |
| Water for Injection* | Solvent | q.s. to 1.0 mL |

*Nitrogen gas is used for WFI sparging (0.01 ppm dissolved Oxygen) and Argon gas is used for overlay (≦1% headspace Oxygen) to prevent oxidation and discoloration of formulated product

### Example 3

The third formulation (Table 11) is comprised of 50 mM TRIS-Hydrochloride buffer, pH range of 8.8-9.6 (target pH 9.2), 18-20 mg/mL of acadesine and WFI. Nitrogen sparging improves stability and reduces discoloration. Overlay of this formulation is desirable to minimize residual headspace oxygen level below ambient (preferably below 15%). An increase in pH allows to provide a shelf-life of 2-3 years for acadesine injectable solution without strict requirement for headspace control.

TABLE 11
Acadesine Injectable Solution, 18-20 mg/mL pH range 8.8-9.6,
target H 9.2

| Ingredients of Acadesine | | |
|---|---|---|
| Solution | Function | Concentration (mg/mL) |
| Acadesine (API) | Active | 18.00-20.00* |
| Tromethamine (TRIS base) | Buffering agent | 6.06 |
| Hydrochloric Acid | Buffering agent | 0.43 |
| Water for Injection** | Solvent | q.s. to 1.0 mL |

[symbol]

Available solubility data supports up to 21.5 mg/mL at 5° C. [symbol]
*Nitrogen gas is used for WFI sparging (0.01 ppm dissolved oxygen) and for overlay (≦15% headspace Oxygen) to prevent oxidation and discoloration of formulated product [symbol]
indicates data missing or illegible when filed

### Example 4

Table 12 illustrates that argon gas is more preferable to nitrogen gas for headspace control. Use of argon gas overlay (in head space) consistently produced lower levels of the residual oxygen (˜1.5 fold less) in comparison to nitrogen gas.

TABLE 12
Nitrogen vs Argon overlay performance, comparison of vial fill processes
for acadesine injectable solution, 18 mg/mL

| | |
|---|---|
| | Vial Headspace |
| | (average of |
| | multiple vials) |
| Overlay duration, | % oxygen |

| Gas flow (SCFH) | seconds/vial | Nitrogen | Argon |
|---|---|---|---|
| 5 | 5 | 2.4 | 1.6 |
| 5 | 10 | 3.0 | 1.7 |
| 5 | 20 | 2.8 | 1.7 |

All of the embodiments and examples herein are in no way intended to limit the scope of the instant invention. Further, it can be appreciated by one of ordinary skill in the art that many changes and modifications can be made thereto without departing from the spirit or scope of the appended claims, and such changes and modifications are contemplated within the scope of the instant invention.

1. A buffered solution comprising acadesine (5-Aminoimidazole-4-Carboxamide Riboside), or a pharmaceutically acceptable salt thereof, wherein said buffered solution has a pH of 6.0-9.6, and a headspace oxygen content of ≦15%, and wherein said buffered solution is substantially free of discoloration, said buffered solution being characterized by having less than or equal to (≦) 5 APHA units.

2. The buffered solution of claim 1, wherein the buffer is selected from the group consisting of: citrate, phosphate, and TRIS-Hydrochloride.

3. The buffered solution of claim 2, wherein the buffer is TRIS-Hydrochloride.

4. The buffered solution of claim 1, wherein the buffer strength ranges from 10 mM to 100 mM.

5. The buffered solution of claim 1, wherein the buffer strength is 50 mM.

6. The buffered solution of claim 1, further comprising a solubilizer selected from the group consisting of polyethylene glycol and propylene glycol.

7. The buffered solution of claim 1, having a pH of 7.2-8.0.

8. The buffered solution of claim 7, having ≦0.01 ppm dissolved oxygen and ≦1% headspace oxygen, comprising:
a) 1.7-6.0 wt % acadesine, or equivalent acadesine amount in pharmaceutically acceptable salt form;
b) 0.55-0.65 wt % tromethamine (TRIS base);
c) 0.30-0.50 wt % concentrated hydrochloric acid (37% HCl); and
d) 92.8-97.5 wt % of water.

9. The buffered solution of claim 8, wherein said dissolved oxygen content is achieved by the use of nitrogen gas sparging.

10. The buffered solution of claim 8, wherein said headspace oxygen content is achieved by the use of nitrogen gas or argon gas overlay.

11. The buffered solution of claim 10, wherein said headspace oxygen content is achieved by the use of argon gas overlay.

12. The buffered solution of claim 8, that is terminally sterilizable at 121° C. up to 40 minutes.

13. (canceled)

14. The buffered solution of claim 8, comprising:
a) 1.79 wt % acadesine or equivalent acadesine amount in pharmaceutically acceptable salt form;
b) 0.60 wt % tromethamine (TRIS base);
c) 0.32-0.47 wt % concentrated hydrochloric acid (37% HCl); and
d) 97.1-97.3 wt % of water.

15. The buffered solution of claim 8, comprising:
a) 5.96 wt % acadesine or equivalent acadesine amount in pharmaceutically acceptable salt form;
b) 0.60 wt % tromethamine (TRIS base);
c) 0.32-0.47 wt % concentrated hydrochloric acid (37% HCl); and
d) 97.97-93.12 wt % of water.

16. The buffered solution of claim 8, having an acadesine concentration of about 18-60 mg of acadesine per mL of solution, or equivalent acadesine concentration in pharmaceutically acceptable salt form.

17. The buffered solution of claim 14, having an acadesine concentration of about 18 mg of acadesine per mL of solution, or equivalent acadesine concentration in pharmaceutically acceptable salt form.

18. The buffered solution of claim 15, having an acadesine concentration of about 60 mg of acadesine per mL of solution, or equivalent acadesine concentration in pharmaceutically acceptable salt form.

19. The buffered solution of claim 1, having a pH of 6.0-8.0 and further comprising an antioxidant.

20. (canceled)

21. The buffered solution of claim 1, having a pH of 8.8-9.6.

22. An acadesine composition comprising an admixture of the buffered solution of claim 1 and an aqueous solution distinct from said buffered solution of claim 1, said aqueous solution selected from the group consisting of normal saline solution, dextrose solution, and cardioplegic solution.

23. A method of treating or inhibiting ischemic conditions regulated by adenosine or reduced blood flow to a tissue or to delay morbidity or death in a patient comprising administering to the patient an effective amount of the buffered solution of claim 1.

24. The method of claim 23, where said condition is selected from the group consisting of a heart attack, a stroke, a transmural or non-transmural myocardial infarction, an acute myocardial infarction, coronary artery disease, coronary heart disease, an arrhythmia, sudden cardia death, a cerebrovascular accident, congestive heart failure, a life-threatening dysrhythmia, cardiomyopathy, a transient ischemic attack, angina pectoralis, the microvascular disease of diabetes mellitus, acute bowel ischemia, kidney ischemia, intermittent claudication of skeletal muscle, migraine headaches, Raynaud's phenomenon, a hepatic injury, a pancreatic injury, and disseminated intravascular coagulation such as due to bowel ischemia, shock or a combination thereof.

25. A kit comprising: a container comprising the buffered solution of claim 1, and instructions for use thereof in treating or inhibiting/ameliorating a condition in a patient.

26. The kit of claim 25, wherein said condition is selected from the group consisting of a heart attack, a stroke, a transmural or non-transmural myocardial infarction, an acute myocardial infarction, coronary artery disease, coronary heart disease, an arrhythmia, sudden cardia death, a cerebrovascular accident, congestive heart failure, a life-threatening dysrhythmia, cardiomyopathy, a transient ischemic attack, angina pectoralis, the microvascular disease of diabetes mellitus, acute bowel ischemia, kidney ischemia, intermittent claudication of skeletal muscle, migraine headaches, Raynaud's phenomenon, a hepatic injury, a pancreatic injury, and disseminated intravascular coagulation such as due to bowel ischemia, shock or a combination thereof.
